(19) United States
(12) Patent Application Publication
(10) Pub. No.: US 2003/0147826 A1
(43) Pub. Date: Aug. 7, 2003

(54) Composition and a process for the biocidal treatment of surfaces

(76) Inventors: Olivier Anthony, Highland Park, NJ (US); Mathieu Joanicot, Lawrenceville, NJ (US)

Correspondence Address: Rhodia Inc., 259 Prospect Plains Road, CN 7500, Cranbury, NJ, 08512, US

(21) Appl. No.: 10/340,441

(22) Filed: Jan. 10, 2003

Related U.S. Application Data
(63) Continuation of application No. 09/590,117, filed on Jun. 8, 2000.

(51) Int. Cl. A61K 7/06; A61K 7/11; A61K 7/075; A61K 7/08

(52) U.S. Cl. 424/70.11; 424/70.28

(57) ABSTRACT

Aqueous composition and method for the biocidal treatment of surfaces, the said composition comprising: (a) water, (b) a cationic surfactant having a biocidal effect and/or a cationic polymer having a biocidal effect, (c) a film-forming polymer forming a transparent film, (d) at least one water-soluble block copolymer comprising at least one anionic hydrophilic block and at least one nonionic hydrophilic block, and, (e) optionally, a nonionic surfactant. Use of the composition as a biocidal composition with a persistent effect for the treatment of surfaces, the biocidal effect on the surface to be treated being retained after several rinses.

The subject of the present invention is a composition with delayed or persistent biocidal effect and a method of biocidal treatment of skin, or keratinous, as well as hard industrial, domestic or community surfaces.

The present invention also relates to a method of using the said aqueous biocidal composition for the treatment and the disinfection, with lasting effect, of skin surfaces, as well as of hard industrial, domestic or community surfaces, by a slow, gradual release of the said biocide after application, upon contact between water and the treated surface.

Aqueous biocidal compositions for the treatment of hard surfaces generally have the disadvantage of rapidly losing their efficacy after their application, especially when the treated surfaces are then washed. To overcome this disadvantage, it has been proposed to use in these compositions film-forming organic polymers in order to form, after application, a physical barrier which makes it possible to combat the excessively rapid release of the biocide. An illustration of this technique (WO 97/06675 by Rhône-Poulenc Chemicals Ltd.) consists in combining a biocide with a terephthalic copolyester having in its polymer chain polyoxyethylene or polyoxyethyleneterephthalate units.

Moreover, an aqueous system based on a biocide and a polyorganosiloxane with polyether functions as well as its use for the disinfection, with lasting effect, of hard surfaces by a slow, gradual release of the said biocide after application, upon contact between water and the treated surface, have been proposed (WO 99/18784 by Rhodia Chimie).

The applicant has found an aqueous biocidal composition with higher efficiency. Indeed, this composition, when applied to the surface to be treated and after evaporation of the water, has an effective biocidal action, even after several rinses of the said treated surface with water.

A first subject of the invention consists in a composition with delayed or persistent biocidal effect comprising:

(a) water,

(b) a cationic surfactant having a biocidal effect,

(c) a film-forming polymer forming a transparent film, and

(d) at least one water-soluble block copolymer comprising at least one anionic hydrophilic block and at least one nonionic hydrophilic block.

A second subject of the invention consists in a method of biocidal treatment of skin surfaces, as well as of hard industrial, domestic or community surfaces, in order to confer on the said surfaces a biocidal effect which is persistent even after several washes or rinses, by applying to the said surfaces a quantity with effective biocidal effect of an aqueous composition with persistent biocidal effect in accordance with the invention, followed by evaporation of the water. This evaporation of the water may be carried out at room temperature, which is the case in particular for detergent compositions for the washing of clothes by hand, for the rinsing of clothes, for body hygiene compositions and for the cleaning and disinfection of hard industrial, domestic or community surfaces. This evaporation may be carried out at a temperature higher than room temperature, which is the case for example for clothes treated and dried in a tumble drier.

During subsequent stages of the method, an effective quantity of the biocide is released on each rinsing, with water, of the surface to be treated. The number of effective rinses can vary from a few units to several tens. Other methods exist for applying the composition. One of these methods consists in precipitating the constituents (b), (c) and (d) of the composition at the surface to be treated without evaporating the water, this water then being removed simply by flowing.

Compound (b) is a cationic surfactant with a biocidal effect. This compound (b) may also be a polymer with a biocidal effect and, in this case, it is not necessary for this polymer to also possess surfactant properties. Compound (b) can therefore be a chemical product consisting of a surfactant portion and of another portion with a biocidal effect. As examples of product (b), there may be mentioned:

the quaternary monoammonium salts of formula:

R¹R²R³R⁴N⁺X⁻

where

R¹ represents a benzyl group optionally substituted with a chlorine atom or a C₁-C₄ alkylbenzyl group, R² represents a C₈-C₂₄ alkyl group,

R³ and R⁴, which are similar or different, represent a C₁-C₄ hydroxyalkyl or alkyl group, and

X⁻ is a solubilizing anion such as halide (for example chloride, bromide or iodide), sulphate or methyl sulphate;

the products of formula:

R^{1′}R^{2′}R^{3′}R^{4′}N⁺X⁻

where

R^{1′} and R^{2′}, which are similar or different, represent a C₈-C₂₄ alkyl group,

R^{3′} and R^{4′}, which are similar or different, represent a C₁-C₄ alkyl group, and

X⁻ is a solubilizing anion such as halide (for example chloride, bromide or iodide), sulphate or methyl sulphate; and

the products of formula:

R^{1″}R^{2′}R^{4″}N⁺X⁻

where

R¹ represents a C₈-C₂₄ alkyl group,

R^{2″}, R^{3″} R^{4″}, which are similar or different, represent a C₁-C₄ alkyl group, and

X⁻ is a solubilizing anion such as halide (for example chloride, bromide or iodide), sulphate or methyl sulphate.

There may be mentioned more particularly the chlorides of cocoalkylbenzyldimethylammonium, of (C₁₂-C₁₄ alkyl)benzyldimethylammonium, of cocoalkyldichlorobenzyldimethylammonium, of tetradecylbenzyldimethylammonium, of didecyldimethylammonium or of dioctyldimethylammonium.

The monoquaternary heterocyclic amine salts such as the chlorides of laurylpyridinium, of cetylpyridinium, of (C₁₂-C₁₄ alkyl)benzylimidazolium, as well as the (fatty alkyl)triphenylphosphonium salts such as myristyltriphenylphosphonium bromide are also suitable.

Also suitable are the cationic polymeric biocides with no notable surfactant properties such as those obtained from the reaction:

of epichlorohydrin and of dimethylamine or of diethylamine

of epichlorohydrin and of imidazole

of 1,3-dichloro-2-propanol and of dimethylamine

of 1,3-dichloro-2-propanol and of 1,3-bis(dimethylamino)-2-propanol

of ethylene dichloride and of 1,3-bis(dimethylamino)-2-propanol

of bis(2-chloroethyl) ether and of N,N′-bis(dimethylaminopropyl)urea or thiourea.

A polymeric biocide which is more particularly suitable is the product of the reaction of dimethylamine with epichlorohydrin and marketed in particular under the name GLOKILL by the company RHODIA. This polymer has units of formula:

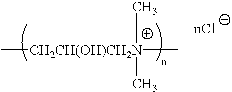

Where n is an integer whose value confers on the polymer a weight-average molecular mass of between 1000 and 20,000, generally of between 5000 and 11,000.

Compound (c) is a film-forming polymer forming a transparent film after evaporation of the water contained in the composition deposited on the surface to be treated. As compound (c), it is recommended to use preferably anionic guar. An anionic guar is obtained by reacting a natural guar with a base and then with monochloroacetate in order to obtain a carboxymethyl guar. Examples of anionic guar which can be used in the context of the present invention are marketed under the trade names GALAXY 707D® by the company Aqualon, and JAGUAR 800® or JAGUAR 8707® marketed by the company Hi Tek.

Compound (d) is a water-soluble block copolymer comprising at least one anionic hydrophilic block and at least one nonionic hydrophilic block. These copolymers have a number-average molecular mass generally preferably of between 2000 and 20,000, preferably between 4000 and 10,000 g/mol.

The anionic blocks include, for example, polymethacrylic acid and its salts, polyacrylic acid and its salts, the copolymers of methacrylic acid and its salts, the copolymers of acrylic acid and its salts, heparin, polyphosphate, polyamino acids such as polyaspartic acid, polyglutaminic acid, polymalic acid, polylactic acid. The preferred anionic blocks in the context of the present invention are the blocks having carboxyl groups in the polymer chain. Examples of monomers which make it possible to prepare such blocks are acrylic acid, aspartic acid, citraconic acid, p-hydroxycinnamic acid, transglutaconic acid, glutamic acid, itaconic acid, linoleic acid, methacrylic acid, maleic acid, oleic acid, maleic anhydride, mesaconic acid, 2-propene-1-sulphonic acid and vinylsulphonic acid.

The nonionic blocks include, for example, polyether glycols, in other words polyethylene oxide, polypropylene oxide, copolymers of ethylene oxide and propylene oxide, polysaccharides, polyacrylamides, polyacrylic esters, polymethacrylamide, polyvinyl alcohol, polyvinylpyrrolidone, polyortho esters, polyamino acids and polyglycerols. To prepare the block polymers, it is possible for example to use anionic polymerization with sequential addition of 2 monomers as described for example by Schmolka, J. Am. Oil Chem. Soc. 1977, 54, 110; or alternatively Wilczek-Veraet et al., Macromolecules 1996, 29, 4036. Another method which can be used consists in initiating the polymerization of a block polymer at each of the ends of another block polymer as described for example by Katayose and Kataoka, Proc. Intern. Symp. Control. Rel. Bioact. Materials, 1996, 23, 899.

In the context of the present invention, it is recommended to use living or controlled polymerization as defined by Quirk and Lee (Polymer International 27, 359 (1992)). Indeed, this particular method makes it possible to prepare polymers with a narrow dispersity and in which the length and the composition of the blocks are controlled by the stoichiometry and the degree of conversion. In the context of this type of polymerization, there are more particularly recommended the block copolymers which can be obtained by any so-called living or controlled polymerization method such as, for example:

the controlled free-radical polymerization by the xanthates according to the teaching of application WO 98/58974,

the controlled free-radical polymerization by the dithioesters according to the teaching of application WO 97/01478,

the polymerization with the aid of nitroxide precursors according to the teaching of application WO 99/03894,

the controlled free-radical polymerization by the dithiocarbamates according to the teaching of application WO 99/31144,

the atom transfer free-radical polymerization (ATRP) according to the teaching of application WO 96/30421,

the controlled free-radical polymerization by the initiators according to the teaching of Otu et al., Makromol. Chem. Rapid. Commun., 3, 127 (1982),

the controlled free-radical polymerization by degenerative transfer of iodine according to the teaching of Tatemoto et al., Jap. 50, 127, 991 (1975), Daikin Kogyo Co Ltd Japan and Matyjaszewski et al., Macromolecules, 28, 2093 (1995)),

the group transfer polymerization according to the teaching of Webster O. W., “Group Transfer Polymerization”, p. 580-588 from the “Encyclopedia of Polymer Science and Engineering”, vol. 7 and H. F. Mark, N. M. Bikales, C. G. Overberger and G. Menges, Eds., Wiley Interscience, New York, 1987,

the controlled free-radical polymerization by the derivatives of tetraphenylethane (D. Braun et al. Macromol. Symp. 111, 63 (1996)),

the controlled free-radical polymerization by the organocobalt complexes (Wayland et al., J. Am. Chem. Soc. 116, 7973 (1994)).

The composition according to the invention may, in addition, optionally comprise a nonionic surfactant (e). As nonionic surfactant (e), there may be mentioned in particular the condensates of alkylene oxide having from 1 to 4 carbon atoms, in particular of ethylene oxide with alcohols, polyols, alkylphenols, fatty acid esters, fatty acid amides and fatty amines; the amine oxides, the sugar derivatives such as alkyl polyglycosides and the esters of fatty acids and sugars, in particular sucrose monopalmitate; the long-chain tertiary phosphine oxides; the dialkyl sulphoxides; the block copolymers of polyoxyethylene and polyoxypropylene; the polyalkoxylated esters of sorbitan; the fatty esters of sorbitan; the polyethylene oxides and amides of fatty acids modified so as to confer on them a hydrophobic character (for example the mono- and diethanolamides of fatty acids containing from 10 to 18 carbon atoms).

As compound (b) which can be used in the compositions according to the invention, there may also be mentioned:

the polyoxyalkylenated (polyethoxyethylenated, polyoxypropylenated or polyoxybutylenated) alkylphenols in which the alkyl substituent is a C₆-C₁₂ and which contain from 5 to 25 oxyalkylene units; by way of example, there may be mentioned TRITON X-45, X-114, X-100 or X-102 which are marketed by Rohm & Haas Co.;

the glucosamides, glucamides or glycerolamides;

the polyoxyalkylenated C₈-C₂₂ aliphatic alcohols containing from 1 to 25 oxyalkylene (oxyethylene or oxypropylene) units. By way of example, there may be mentioned TERGITOL 15-S-9, TERGITOL 24-L-6 NMW which are marketed by Union Carbide Corp., NEODOL 45-9, NEODOL 23-65, NEODOL 45-7, NEODOL 45-4 which are marketed by Shell Chemical Co., RHODASURF ID060, RHODASURF LA90, RHODASURF IT070 which are marketed by the company RHODIA;

the amine oxides such as the (C₁₀-C₁₈ alkyl)dimethylamine oxides, the (C₈-C₂₂ alkoxy)ethyldihydroxy-ethylamine oxides;

the alkylpolyglycosides, more particularly those described in U.S. Pat. No. 4,565,647;

the C8-C₂₀ fatty acid amides;

the ethoxylated fatty acids; and

the ethoxylated amines.

The compositions according to the invention are provided in the form of a single-phase aqueous solution of all its constituents, which is a desired property difficult to obtain for compositions of this type. Without wishing to limit the invention to a particular scientific theory, the applicant is of the opinion that compound (b) and compound (a) combine into aggregates or micelles whose size is of the order of a few tens of nanometers. The core of the aggregate would consist of a coalesced product of the anionic portion (d) with compound (b). These aggregates have a sufficient size to remain trapped in the network of the film-forming polymer (c) after application of the solution and evaporation of the water. The particulate structure of this coalesced product ensures a slow and appropriate release of the biocide during rinses.

Preferably, the compositions according to the present invention comprise:

(a) 100 parts by weight of water,

(b) 0.05 to 5 parts by weight of a cationic surfactant having a biocidal effect,

(c) 0.02 to 1 part by weight of a film-forming polymer forming a transparent film,

(d) 0.05 to 5 parts by weight of at least one water-soluble block copolymer comprising at least one anionic hydrophilic block and at least one nonionic hydrophilic block, and

(e) optionally, 1 to 20 parts by weight of a nonionic surfactant.

The compositions more preferred still according to the present invention comprise:

(a) 100 parts by weight of water,

(b) 0.5 to 2 parts by weight of a cationic surfactant having a biocidal effect,

(c) 0.05 to 0.3 parts by weight of a film-forming polymer forming a transparent film,

(d) 0.5 to 2 parts by weight of at least one water-soluble block copolymer comprising at least one anionic hydrophilic block and at least one nonionic hydrophilic block, and

(e) optionally, 3 to 8 parts by weight of a nonionic surfactant.

The choice of the nature of the biocidal surfactant(s) used depends on the desired application (body hygiene, disinfection of various hard surfaces).

The quantity of biocidal agent (b) used depends on the field of application for which the method of the invention is used.

Thus, in the field of body hygiene for the hair or the skin, the quantity of biocide (b) used is generally of the order of 0.05 to 0.3% in order to comply with the current legislation.

The biocidal composition used for carrying out the method of the invention may be of a different type, depending on the field of application for which the method of the invention is used.

It may be a body hygiene composition for the hair or the skin, in particular in the form of shampoos, lotions, shower gels for the hair or the body, or liquid soaps for the face or the body.

It may also include detergent compositions for the cleaning and disinfection of hard industrial, domestic or community surfaces.

According to the invention, besides the main constituents of the aqueous biocidal composition, other constituents may be present whose nature depends on the field of application for which the method of the invention is used.

Thus, biocidal compositions for the treatment of hard surfaces may, in addition, comprise additives such as chelating agents such as aminocarboxylates (ethylenediaminetetraacetates, nitrilotriacetates, N,N-bis(carboxymethyl)glutamates, citrates), alcohols (ethanol, isopropanol, glycols), detergency adjuvants (phosphates, silicates), surfactants, colorants, perfumes and the like.

Compositions for hair and skin hygiene may, in addition, contain surfactants, humectants, emollients, viscosity-promoting or gelling agents, sequestering agents, conditioners, colorants, perfumes and the like.

The said method of biocidal treatment of surfaces which is the subject of the invention may be used to carry out the treatment of keratinous or skin body surfaces, of various hard surfaces such as tiles, floors, walls, work surfaces, equipment, furniture, instruments and the like, in industry, the agri-foodstuffs sector, the domestic sectors (kitchens, bathrooms, toilets and the like) and in community places.

Among the hard surfaces which can be treated, there may be mentioned in particular those made of ceramic, porcelain, glass, polyvinyl chloride, formica or other hard organic polymer, stainless steel, aluminium, wood and the like.

The disinfection operation consists in applying or bringing the said aqueous biocidal composition, optionally diluted 1- to 1000-fold, preferably 1- to 100-fold, to or into contact with the surface to be treated, and then in allowing the water to evaporate.

Among the microorganisms whose proliferation can be controlled using the method of the invention, there may be mentioned

Gram-negative bacteria such as: Pseudomonas aeruginosa; Escherichia coli; Proteus mirabilis

Gram-positive bacteria such as: Staphylococcus aureus; Streptococcus faecium

other bacteria which are dangerous in food such as: Salmonella typhimurium; Listeria monocytogenes; Campylobacter jejuni; Yersinia enterocolitica

yeasts such as: Saccharomyces cerevisiae; Candida albicans

fungi such as: Aspergillus niger; Fusarium solani; Pencillium chrysogenum

algae such as: Chlorella saccharophilia; Chlorella emersonii; Chlorella vulgaris; Chlamydomonas eugametos

The following examples illustrate the invention without limiting the scope thereof.

### EXAMPLE 1

Synthesis of a Polyethyl Acrylate-b-polyvinyl Acetate Diblock Copolymer (PAEt-b-PVAc):

1411 g of water, 0.88 g of sodium carbonate and 23.98 g of sodium dodecyl sulphate are introduced at 30° C. into a jacketed 4-1 reactor provided with a stirring blade. The reactor is purged with a nitrogen stream and heated up to 85° C., with stirring. During the rise in temperature, at 80° C., 1.63 g of methacrylic acid, 31.71 g of ethyl acrylate and 17.5 g of S-propionyl O-ethyl xanthate are introduced. A solution of 1.34 g of ammonium persulphate in 2.67 g of water is introduced at 85° C. Next, a mixture of 285.48 g of ethyl acrylate and 15.03 g of methacrylic acid is added continuously over one hour. The system is maintained at 85° C. for an additional 45 minutes, after which time a sample is removed for GC analysis (vapour phase chromatography and Mn: number-average molecular mass, M_{w}: weight-average molecular mass):

Mₙ=4380 g/mol

M_{w}/Mₙ=1.7

By maintaining the reactor at 85° C., the synthesis of the diblock copolymer is carried out by adding 1088 g of vinyl acetate over 2 h 15 minutes, as well as a mixture of 60 g of water, 1.26 g of sodium carbonate and 0.35 g of ammonium persulphate over the same period. At the end of the introduction, 0.8 g of ammonium persulphate are added and the reactor is maintained at 85° C. for an additional two hours. The reactor is then cooled. A sample is removed for analysis:

Mₙ=20200 g/mol

M_{w}/Mₙ=1.58

The presence of the xanthate group at the chain end is confirmed by GC (UV detection at 290 nm).

### EXAMPLE 2

Synthesis of a Polyacrylic Acid-b-polyvinyl Alcohol Diblock Copolymer (PAA-b-PVA) by Basic Hydrolysis of the PAEt-b-PVAc Copolymer Described in Example 1:

The hydrolysis of the PAEt-b-PVAc copolymer described in Example 1 is carried out in the reactor for the synthesis of the diblock polymer used in Example 1 on an equivalent of 400 g of dry matter (that is to say 1045 g of latex at 38.25% of dry extract). The pH of the latex is adjusted to 8 with a 1N sodium hydroxide solution. The reactor is heated at 60° C. and maintained under a nitrogen stream. 1115 g of 4N sodium hydroxide are added over 1 hour, with vigorous stirring. The system is maintained at this temperature for 11 hours. The final dry extract is 16.25%. A sample collected at the end of the reaction is analysed by ¹H NMR. The analysis confirms the disappearance of the peaks characteristic of the acrylic esters and of the acetate group.

### EXAMPLE 3

A sodium polyacrylate/polyvinylalcohol diblock copolymer synthesized according to Example 2 is used.

The polyvinylalcohol and sodium polyacrylate blocks contain 184 polyvinylalcohol monomers and 44 sodium acrylate monomers respectively. The copolymer is then purified in the following manner: a solution of the copolymer at 5% by weight in water is precipitated dropwise in a large excess of acetone and then redissolved in water. This cycle is performed twice in order to obtain a very transparent pale yellow solution. The pH is then adjusted to 10 by addition of sodium hydroxide. The number-average molecular mass of this polymer is 20,200.

The biocide used is benzyldodecyldimethylammonium.

The isotherm for the attachment of the benzyldodecyldimethylammonium chloride to the sodium polyacrylate/polyvinylalcohol diblock copolymer was determined by potentiometry with the aid of an electrode specific for this cationic surfactant. The results obtained are assembled in Table 1 below. The electrode is the following: Ag/AgCl|3M NH₄Cl agar bridge; the reference solution is a solution of biocide a at 3.10⁻⁴ Mol and the membrane is a PVC membrane. The preparation and the assembling of the electrode are described elsewhere (Shirahama and Tashiro, Bull. Chem. Soc. Jpn., 1984, 57, 377; Benrraou et al, J. Phys. Chem., 1992, 96, 1468) The electromotive force (EMF) was measured with the aid of a VWR Scientific model 8015 multimeter. This electrode has a nerstian response over the whole concentration domain studied. Two benzyldodecyldimethylammonium chloride solutions at concentrations of 0.1 and 1% by weight were gradually added to 30 ml of copolymer at a concentration of 0.10% by weight. The variation in diblock concentration following the addition of surfactant was taken into account. The fraction of binding sites occupied (β) is determined as follows:

β=(Cₜₒₜ−Cₗ)/Cₛ

where Cₜₒₜ is the total concentration of surfactant added, Cₗ the concentration of free surfactant, not attached to the polymer, and Cₛ the concentration of binding sites on the polymer, that is to say the concentration of charges. C_{f} is determined with the aid of a calibration curve established beforehand in the absence of copolymer. As indicated in Table 1, the appearance of a slight cloudiness is observed for β values close to 1, that is to say when the charges of the surfactants attached compensate for the charges present on the acrylic acid block of the diblock copolymer.

### COMPARATIVE EXAMPLE

By way of comparison, the procedure of Example 3 is exactly repeated except that the diblock copolymer is replaced with an acrylic acid homopolymer comprising 111 acrylic acid units per polymer chain. The pH was also adjusted to 10 with sodium hydroxide (Table 1). The polymer concentration was adjusted so that the concentration of sodium acrylate units is equal to that used in the experiment carried out with the diblock, that is to say 0.03% by weight.

It is observed that the critical aggregation concentration (cac), that is to say the minimum concentration above which the attachment of the surfactant to the polymer, of the benzyldodecyldimethylammonium chloride, is substantially the same as for the diblock copolymer: about 9×10⁻⁴% by weight. On the other hand, the solution becomes slightly cloudy for a P value close to 0.1 and a macroscopic precipitation is observed for a β value close to 0.8, these two phenomena not appearing with the diblock.

TABLE 1
Isotherm for the attachment of benzyldodecyldimethyl-
ammonium chloride on the sodium polyacrylate/polyvinyl
alcohol diblock copolymer (copolymer) and on a sodium
acrylate homopolymer (PAA)

| [RP50] free, | PAA 8 K | [RP50] free, | Rhodibloc 015 |
|---|---|---|---|
| wt. % | Beta | wt. % | Beta |
| 5.41E−04 | 0.0004 | 7.69E−04 | 0.0006 |
| 6.45E−04 | 0.0016 | 8.49E−04 | 0.0018 |
| 6.96E−04 | 0.0030 | 9.09E−04 | 0.0036 |
| 7.32E−04 | 0.0046 | 9.82E−04 | 0.0064 |
| 7.87E−04 | 0.0069 | 1.04E−03 | 0.0101 |
| 8.34E−04 | 0.0098 | 1.10E−03 | 0.0150 |
| 8.38E−04 | 0.0136 | 1.17E−03 | 0.0238 |
| 8.52E−04 | 0.0185 | 1.25E−03 | 0.0364 |
| 8.56E−04 | 0.0241 | 1.35E−03 | 0.0566 |
| 8.70E−04 | 0.0328 | 1.48E−03 | 0.0819 |
| 8.78E−04 | 0.0427 | 1.60E−03 | 0.1202 |
| 8.81E−04 | 0.0552 | 1.80E−03 | 0.1839 |
| 8.93E−04 | 0.0801 | 2.02E−03 | 0.2862 |
| 9.15E−04 | 0.10497 (1) | 2.46E−03 | 0.4391 |
| 9.43E−04 | 0.1361 | 3.27E−03 | 0.6933 |
| 9.71E−04 | 0.1860 | 6.01E−03 | 0.8525 |
| 9.83E−04 | 0.2610 | 1.66E−02 | 1.0124 (1) |
| 1.20E−03 | 0.3352 | 2.92E−02 | 1.0738 |
| 1.38E−03 | 0.4097 | | |
| 1.71E−03 | 0.5337 | | |
| 2.21E−03 | 0.6571 | | |
| 3.99E−03 | 0.77447 (2) | | |
| 1.11E−02 | 0.8659 | | |

(1) Turbidity limit
(2) Macroscopic precipitation limit

### EXAMPLE 4

The diblock copolymer and the biocidal cationic surfactant described in Example 3 are again used in a more concentrated formulation. A solution of 1.5% by weight of cationic surfactant and of 1% by weight of diblock copolymer is prepared. The appearance of a white precipitate consisting of the copolymer-surfactant complex is observed. This complex is completely redissolved by the addition of a total concentration of 5% by weight of nonionic surfactant (RHODASURF DA639, an isododecyl ether hexaoxyethylene glycol sold by the company Rhodia). This redissolution shows the co-micellization of the cationic and neutral surfactants on the polymer. The anionic surfactant provides, by attaching to the complex, a hydrophilic character sufficient to avoid phase separation.

An aqueous solution of anionic guar (carboxymethyl guar, CMG, having a molecular mass of 2.3 million and a degree of anionic substitution of 0.10) is then added. The final concentration of carboxymethyl guar is 0.15%. The addition of this polymer does not induce phase separation in the solution.

A single-phase detergent solution is thus obtained.

### EXAMPLE 5

The cationic and nonionic surfactants as well as the block copolymer and the anionic guar are identical to those of Example 4.

A bathroom tile is cut into 5*5 cm squares. Only the smooth and impermeable side of the tile will be used in this study. A silicone coating which polymerizes upon contact with the air is deposited on the edges of each square placed flat, the smooth face facing up. After 24 hours, this coating forms a very resistant rubber which makes it possible to avoid the flow of a liquid containing, inter alia (see description below), benzyldodecyldimethylammonium chloride on the surface of the smooth side of the tile. 3 ml of a given liquid formula are then deposited on it. The tile and the liquid deposited at its surface are finally placed for 24 hours in a laminar flow cabinet, until there is completely drying and formation of a uniform film at the surface.

This tile is then vertically suspended over a beaker containing 150 ml of an aqueous solution of sodium chloride having a concentration of 0.5 M.

The surface covered with the dry film is then sprayed with the aid of a vaporizer. Each spraying discharges 1 ml of water over the surface. The liquid falling from the surface is recovered in the beaker and 2.5 ml are collected in the beaker after each spraying.

The concentration of benzyldodecyldimethylammonium chloride is determined by measuring the UV absorbance of the solution collected at 262.5 nm and with reference to a calibration curve established beforehand. A Hitachi model U3410 spectrometer was used for this measurement.

This concentration allows us to calculate the quantity of benzyldodecyldimethylammonium chloride which remained on the tile in spite of the rinse. We took into account the variation in the volume of liquid in the beaker due to the addition of the water sprayed over the tile and to the collection necessary in order to carry out the measurement. The results are assembled in Table 2 below giving the percentage of benzyldodecyldimethylammonium chloride remaining on the surface of the tile as a function of the number of rinses.

TABLE 2
Percentage of benzyldodecyldimethylammonium chloride
remaining on the tile as a function of the number of
rinses.

| | 5 rinses | 10 | 15 | 21 | 30 |
|---|---|---|---|---|---|
| Formula A | 18% | 4% | 3% | NA | NA |
| Formula B | 82% | 48% | 14% | 8% | 6% |
| Formula C | 81% | 65% | 52% | 42% | 30% |

Formula A: 1.5% by weight of benzyldodecyldimethyl-ammonium chloride
Formula B: 1.5% by weight of benzyldodecyldimethyl-ammonium chloride 5% by weight of isododecyl ether hexaoxyethylene glycol 0.15% by weight of anionic guar
Formula C: 1.5% by weight of benzyldodecyldimethyl-ammonium chloride 5% by weight of isododecyl ether hexaoxyethylene glycol 0.15% by weight of anionic guar 1% by weight of diblock copolymer (vinyl alcohol and sodium acrylate block copolymer described in

### EXAMPLE 6

The diblock copolymer described in Example 3 above is again used and as cationic agent with a biocidal effect Glokill is added which is a biocidal quaternary amine polymer which is positively charged at all pH values, a product marketed by the company RHODIA. The structure of the recurring units of this polymeric biocide is the following:

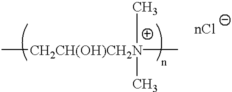

n is an integer whose value confers on the polymer a weight-molecular mass of between 5000 and 11,000.

To confirm the formation of aggregates between Glokill and the diblock copolymer, a series of samples in which the concentration of Glokill is fixed and that of the copolymer varies are prepared in the following manner:

First of all, a parameter z is defined as the ratio between the positive charges of Glokill and the negative charges of the anionic portion of the copolymer. A solution of Glokill at 5.2 g/l was mixed with the solution of copolymer so as to obtain a charge ratio varying between z=0 and z=2.5 and a final Glokill concentration of 1.56 g/l. The (dynamic) light scattering technique was used to measure the size in nanometer (nm) of the agglomerates formed by these two polymers. The results presented in Table 3 below give the size of the objects as a function of z.

TABLE 3

| z | Size (nm) |
|---|---|
| 0 | 15 |
| 0.3 | 60 |
| 0.6 | 80 |
| 1 | 150 |
| 1.5 | 120 |
| 2 | 100 |

### EXAMPLE 7

The diblock copolymer described in Example 3 is again used and as cationic agent with a biocidal effect, the GLOKILL used in Example 6 above is added. The composition also comprises the neutral surfactant and is prepared in the following manner: The nonionic surfactant described in Example 4 (RHODASURF DA639, an isododecyl ether hexaoxyethylene glycol) and GLOKILL are added to the solution of diblock copolymer in order to obtain a solution of 0.6% by weight of diblock, of 0.156% by weight of GLOKILL and of 5% of nonionic surfactant.

A single-phase detergent solution is thus obtained.

1. Composition for the biocidal treatment of surfaces with a persistent effect, the said composition comprising: (a) water, (b) a cationic surfactant having a biocidal effect, (c) a film-forming polymer forming a transparent film, and (d) at least one water-soluble block copolymer comprising at least one anionic hydrophilic block and at least one nonionic hydrophilic block.

2. Composition according to claim 1, characterized in that the composition comprises in addition (e) a nonionic surfactant.

3. Composition according to claim 1, characterized in that the cationic surfactant (b) having a biocidal effect is chosen from:
the quaternary monoammonium salts of formula:
R¹R²R³R⁴N⁺X⁻
where R¹ represents a benzyl group optionally substituted with a chlorine atom or a C₁-C₄ alkylbenzyl group, R² represents a C₈-C₂₄ alkyl group, R³ and R⁴, which are similar or different, represent a C₁-C₄ hydroxyalkyl or alkyl group, and X⁻ is a solubilizing anion such as halide (for example chloride, bromide or iodide), sulphate or methyl sulphate;
the products of formula:
R^{1′}R^{2′}R^{3′}R^{4′}N⁺X⁻
where
R^{1′} and R^{2′}, which are similar or different, represent a C₈-C₂₄ alkyl group,
R³ and R⁴, which are similar or different, represent a C₁-C₄ alkyl group, and
X⁻ is a solubilizing anion such as halide (for example chloride, bromide or iodide), sulphate or methyl sulphate; and
the products of formula:
R^{1″}R^{2″}R^{3″}R^{4″}N⁺X⁻
where
R¹ represents a C₈-C₂₄ alkyl group,
R^{2″R}^{3″R}^{4″}, which are similar or different, represent a C₁-C₄ alkyl group, and
X⁻ is a solubilizing anion such as halide (for example chloride, bromide or iodide), sulphate or methyl sulphate.

4. Composition according to any one of the preceding claims, characterized in that the whole or a portion of compound (b) is replaced with a cationic polymer with a biocidal effect.

5. Composition according to claim 4, characterized in that the said cationic polymer is obtained from the reaction:
of epichlorohydrin and of dimethylamine or of diethylamine,
of epichlorohydrin and of imidazole,
of 1,3-dichloro-2-propanol and of dimethylamine,
of 1,3-dichloro-2-propanol and of 1,3-bis(dimethylamino)-2-propanol,
of ethylene dichloride and of 1,3-bis(dimethylamino)-2-propanol, or
of bis(2-chloroethyl) ether and of N,N′-bis(dimethylaminopropyl)urea or thiourea.

6. Composition according to claim 2, characterized in that the nonionic surfactant (e) is chosen from the condensates of alkylene oxide having from 1 to 4 carbon atoms with alcohols, polyols, alkylphenols, fatty acid esters, fatty acid amides and fatty amines; the amine oxides, the sugar derivatives; the long-chain tertiary phosphine oxides; the dialkyl sulphoxides; the block copolymers of polyoxyethylene and polyoxypropylene; the polyalkoxylated esters of sorbitan; the fatty esters of sorbitan; the polyethylene oxides and amides of fatty acids modified so as to confer on them a hydrophobic character; the polyoxyalkylenated alkylphenols in which the alkyl substituent is a C₆-C₁₂ and which contain from 5 to 25 oxyalkylene units; the glucosamides, glucamides or glycerolamides; the polyoxyalkylenated C₈-C₂₂ aliphatic alcohols containing from 1 to 25 units; the amine oxides, the (C₈-C₂₂ alkoxy)ethyldihydroxyethylamine oxides; the alkylpolyglycosides; the C₈-C₂₀ fatty acid amides; the ethoxylated fatty acids; and the ethoxylated amines.

7. Composition according to any one of the preceding claims, characterized in that compound (c) is an anionic guar gum.

8. Composition according to any one of the preceding claims, characterized in that compound (c) is a carboxymethyl guar.

9. Composition according to any one of the preceding claims, characterized in that copolymer (d) has a number-average molecular mass preferably of between 2000 and 20,000, preferably of between 4000 and 10,000 g/mol.

10. Composition according to claim 9, characterized in that copolymer (d) is prepared by living or controlled polymerization.

11. Composition according to claim 1, characterized in that it comprises:
(a) 100 parts by weight of water,
(b) 0.05 to 5 parts by weight of a cationic surfactant having a biocidal effect,
(c) 0.02 to 1 part by weight of a film-forming polymer forming a transparent film, and
(d) 0.05 to 5 parts by weight of at least one water-soluble block copolymer comprising at least one anionic hydrophilic block and at least one nonionic hydrophilic block.

12. Composition according to claim 2, characterized in that it comprises:
(a) 100 parts by weight of water,
(b) 0.05 to 5 parts by weight of a cationic surfactant having a biocidal effect,
(c) 0.02 to 1 part by weight of a film-forming polymer forming a transparent film,
(d) 0.05 to 5 parts by weight of at least one water-soluble block copolymer comprising at least one anionic hydrophilic block and at least one nonionic hydrophilic block, and
(e) 1 to 20 parts by weight of a nonionic surfactant.

13. Composition according to claim 12, characterized in that it comprises:
(a) 100 parts by weight of water,
(b) 0.5 to 2 parts by weight of a cationic surfactant having a biocidal effect,
(c) 3 to 8 parts by weight of a nonionic surfactant,
(d) 0.05 to 0.3 parts by weight of a film-forming polymer forming a transparent film, and
(e) 0.5 to 2 parts by weight of at least one water-soluble block copolymer comprising at least one anionic hydrophilic block and at least one nonionic hydrophilic block.

14. Composition according to any one of the preceding claims, characterized in that the whole or a portion of compound (b) is replaced with a cationic polymer with a biocidal effect.

15. Method of biocidal treatment of skin, or keratinous, as well as of hard industrial, domestic or community surfaces, comprising the following steps:
a) a biocidal composition as defined in any one of claims 1 to 14 is applied to the said surfaces,
b) the applied composition is dried in order to remove the water, and
c) the said surfaces are washed at least once with water.

16. Method according to claim 15, characterized in that the said composition is diluted 1- to 100-fold, preferably 1- to 1000-fold before use.

17. Method according to either of claims 15 and 16, characterized in that the quantity of aqueous biocidal composition used corresponds to a deposition of biocidal agent (b) of 0.01 to 10 g, preferably of 0.1 to 1 g per m² of surface and to a deposition of copolymer (d) of 0.001 to 2 g, preferably of 0.01 to 0.5 g per m² of surface.

18. Method according to any one of claims 15 to 17, characterized in that the said biocidal composition is a body hygiene composition for the hair or the skin, such as shampoos, lotions, gels for the hair or the body, and liquid soaps for the face or the body.

19. Method according to any one of claims 1 to 18, characterized in that the water-soluble block copolymer d) has a number-average molecular mass Mn of between 2000 and 20,000, preferably of between 3000 and 10,000 g/mol.
